(19) United States
(12) Patent Application Publication
(10) Pub. No.: US 2025/0336543 A1
(43) Pub. Date: Oct. 30, 2025

(54) SYSTEM AND METHOD FOR GENERATING AN INSTRUCTION TO ASSIST A PATIENT

(71) Applicant: ZEMPLEE INC., Los Gatos, CA (US)

(72) Inventors: Aparna Pujar Anthapur, Campbell (CA); Ishanya Anthapur, Los Gatos (CA); YngJaw Chen, San Jose, CA (US)

(21) Appl. No.: 19/191,141

(22) Filed: Apr. 28, 2025

Related U.S. Application Data
(60) Provisional application No. 63/639,041, filed on Apr. 26, 2024.

(51) Int. Cl. G16H 50/30; G16H 10/60; G16H 20/13

(52) U.S. Cl. CPC G16H 50/30; G16H 10/60; G16H 20/13

(57) ABSTRACT

A system and method for generating patient care instructions based on real-time sensor and medical data. The method includes receiving time-stamped sensor data from a sensor network comprising motion, occupancy, and environmental sensors, and receiving medical data associated with a patient, including medical conditions, treatment history, medication data, and biometric data. The sensor data is enriched with room-specific information, and activity pattern data is generated in real time using a pattern recognition model. The activity pattern data includes mobility, sleep patterns, medication adherence, statistical measures, temporal patterns, and correlations with medical data. Anomalies indicating potential health risks are detected by comparing current activity patterns with baseline data. A prediction model, trained on historical patient data, assesses the patient's health and generates care instructions accordingly. The care instructions are securely delivered to patient devices, caregiver applications, or automated medication dispensing systems, enabling timely interventions and continuous patient monitoring.

### PRIORITY INFORMATION

The present application claims priority from U.S. Provisional Patent Application No. 63/639,041 filed on Apr. 26, 2024.

### FIELD OF INVENTION

The present disclosure relates to systems and methods for monitoring and assisting patients, and more particularly to a system and method for generating patient care instructions using real-time sensor data and artificial intelligence.

### BACKGROUND

The global demographic landscape is undergoing a significant transformation, with an increasing proportion of the population entering their elderly years. By 2050, it is anticipated that more than one-fifth of the population will be aged 65 or older, and a substantial portion of these individuals will be living independently. The desire to age in place is paramount among the elderly, yet this independence comes with challenges, especially concerning health, safety, and well-being.

Living alone, elderly individuals face the daily task of managing activities of daily living, household chores, and their overall health. The aging process often brings physical limitations, making them susceptible to fall risks and accidents. Families taking on the responsibility of elderly care are confronted with constant concern for the well-being of their loved ones, particularly in emergencies such as falls or disruptions in mobility.

Current personal tracking and monitoring technologies, though developed with good intentions, present limitations. Wearable devices, such as wristwatches or pendants, may be rendered ineffective if the elderly person is incapacitated and unable to activate the device in case of an emergency. Moreover, adapting to these technologies requires lifestyle changes and ongoing education, adding to the burden.

The prevalence of conditions such as dementia, vision loss, and hearing loss further complicates the use of existing solutions. Elders may resist using devices due to psychological reasons or fear of losing independence, contributing to underreporting of incidents.

Notably, fall incidents often occur during transitions from a static state, especially during nocturnal hours when risks are heightened and conventional devices may not be within reach. Existing solutions fall short during these critical moments, necessitating a more comprehensive and non-intrusive approach to passive monitoring.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for generating patient care instructions is provided. The method includes receiving sensor data, wherein each piece of sensor data is associated with a time stamp, and the sensor data comprises at least one of motion data, occupancy data, and environmental data. The method also includes receiving medical data associated with the patient, the medical data comprising at least one of medical conditions, treatment history, medication data, and biometric data. The method further includes enriching the sensor data by incorporating room-specific information. The method involves generating, in real time, activity pattern data by processing the enriched sensor data and the medical data of the patient by using a pattern recognition model, wherein the activity pattern data comprises the activity data of the patient being indicative of at least one of mobility, sleep patterns, and medication adherence, statistical measures of sensor data, temporal patterns, and correlation between sensor data and medical data. The method also includes detecting an anomaly indicating a potential health risk, wherein the anomaly is detected by comparing the activity pattern data with a baseline activity pattern data of the patient. The method further involves predicting health of the patient based on the anomaly and the activity pattern data by utilizing a prediction model, wherein the prediction model is trained on data from other patients, medical data associated with the patient, to recognize patterns that correlate with health conditions. The method includes generating patient care instructions based on the predicted health of the patient, and delivering the patient care instructions through a secure communication channel to at least one of a patient interface device, a caregiver mobile application, or an automated medication dispensing system.

According to other aspects of the present disclosure, the method may include one or more of the following features. The pattern recognition may comprise segmenting the enriched sensor data into time-windowed data blocks of a predefined duration, computing, for each time-windowed data block, a feature vector that includes statistical measures of the sensor data and correlation measures between the sensor data and the medical data, classifying each feature vector by applying a neural network classifier to produce classification data, wherein the classification data comprises a class label or probability scores corresponding to predefined categories related to the patient's health, and generating activity pattern data that maps the classification data to the corresponding time-windowed data blocks, wherein the activity pattern data represents behaviors of the patient, including mobility, sleep patterns, and medication adherence, and serves as a baseline for detecting anomalies and predicting potential health risks. The neural network classifier may be a deep learning model trained on historical sensor data and medical data to recognize patterns indicative of health conditions, and wherein the neural network classifier produces classification data based on learned patterns of the patient's activity, enabling the detection of deviations from normal behavior. The room specific information may provide insight into activity of the patient in different areas of a home. The method may further comprise creating a natural language summary by processing the pattern data, wherein the natural language summary is generated by a fine-tuned Large Language Model, the LLM model is fine-tuned based on historical sensor data and medical data to produce actionable insights related to patient care. The method may further comprise generating patient care instructions based on the natural language summary, the instructions specifying one or more actions to be performed to assist the patient. The patient care instructions may be generated to specify a recommended course of action based on the natural language summary, the course of action being one of a situation advice to the patient, a modification of a treatment plan, a medication reminder, or an alert for medical assistance.

The sensor data may be received from a sensor network comprising motion sensors configured to detect patient movement, door sensors configured to record entry and exit times from different rooms, seating pressure sensors integrated into chairs configured to monitor sitting duration and frequency, and bed mat sensors configured to track sleep patterns, including sleep duration, restlessness. The patient care instructions may include recommendations based on historical health trends derived from medical data, the health trends including disease progression, symptom flare-ups, or treatment adherence. The delivery of the instructions may be performed using at least one of a voice assistant and a mobile device. The method may further comprise storing the activity pattern data and the natural language summary in a database for future use and refinement of the prediction model. The instructions may be delivered in real-time, the delivery being based on a dynamic analysis of the patient's current status as reflected in the most recent sensor and medical data. The method may further comprise authenticating a caregiver using electronic visit verification (EVV), wherein the EVV captures biometric authentication data, such as a fingerprint or facial recognition data, before delivery of the instruction data. The sensor data may be received from a set of sensors in raw format, wherein each sensor provides its respective data, and wherein the sensor data from the set of sensors is mapped and synchronized into a standardized format, with each piece of standardized sensor data further including a time stamp associated with the corresponding sensor data.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a network diagram of a system for generating patient care instructions, according to aspects of the present disclosure.

FIG. 2 illustrates a flowchart of a data analysis and pattern recognition engine, in accordance with example embodiments.

FIG. 3 illustrates a flowchart of a health prediction engine, according to an embodiment.

FIG. 4 illustrates a flowchart of an instruction generation workflow, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a system and method for generating patient care instructions. The system may utilize sensor data and medical information to monitor patient activities, detect anomalies, and predict potential health risks. By analyzing patterns in the collected data, the system may generate customized care instructions to assist caregivers in providing appropriate support to patients.

In some cases, the system may incorporate various types of sensors to gather data on patient movements, environmental conditions, and physiological parameters. This comprehensive data collection approach may enable a more holistic understanding of the patient's daily activities and health status.

The system may employ advanced data processing techniques, including pattern recognition models and machine learning models, to identify relevant trends and deviations from normal behavior. By comparing current activity patterns with established baselines, the system may detect potential issues that warrant attention.

Based on the analyzed data and detected anomalies, the system may generate tailored care instructions. These instructions may be delivered through secure communication channels to caregivers, healthcare providers, or automated systems. The instructions may provide guidance on various aspects of patient care, including medication management, mobility assistance, and dietary recommendations.

In some cases, the system may adapt its analysis and instruction generation based on feedback and outcomes, allowing for continuous improvement in the quality and relevance of the care instructions provided. This adaptive approach may help ensure that the system remains responsive to changing patient needs and evolving healthcare practices.

By automating the process of monitoring patient activities and generating care instructions, the system may help improve the efficiency and effectiveness of caregiving efforts. This approach may potentially lead to better health outcomes for patients and reduced burden on caregivers and healthcare systems.

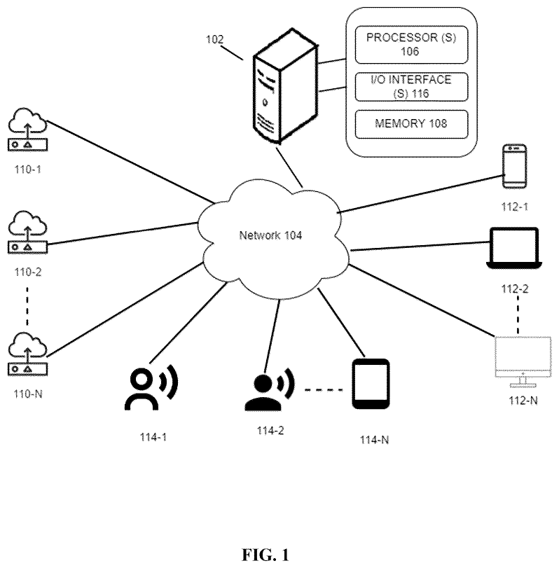

FIG. 1 illustrates a network architecture of a system for generating patient care instructions. The network architecture may include a system 102, a communication network 104, multiple sensors 110-1 to 110-N, multiple guardian devices 112-1 to 112-N, and multiple personal digital assistants 114-1 to 114-N.

A system 102 may be connected to the communication network 104. The system 102 may include a processor 106 and a memory 108. The processor 106 may execute instructions stored in the memory 108 to perform various operations related to data analysis, pattern recognition, and instruction generation.

The communication network 104 may facilitate data exchange between the system 102 and other components of the network architecture. In some cases, the communication network 104 may be a wired or wireless network, or a combination of both.

A first sensor 110-1, a second sensor 110-2, and additional sensors up to an nth sensor 110-N may be connected to the communication network 104. These sensors may collect various types of data related to patient activities and environmental conditions. In some cases, the sensors may be placed in different rooms of a patient's home to provide room-specific information, offering insight into patient activity in different areas of the home.

A first guardian device 112-1, a second guardian device 112-2, and additional guardian devices up to an nth guardian device 112-N may also be connected to the communication network 104. These guardian devices may be used by caregivers or family members to receive patient care instructions and monitor patient activities.

A first personal digital assistant 114-1, a second personal digital assistant 114-2, and additional personal digital assistants up to an nth personal digital assistant 114-N may be connected to the communication network 104. These personal digital assistants may provide an interface for patients to interact with the system or receive care-related information.

In some cases, the network architecture may include an automated medication dispensing system connected to the communication network 104. The automated medication dispensing system may receive patient care instructions from the system 102 and dispense medications accordingly.

The system 102 may receive data from the sensors 110-1 to 110-N, process this data using the processor 106 and memory 108, and generate patient care instructions. These instructions may then be transmitted through the communication network 104 to the appropriate guardian devices 112-1 to 112-N, personal digital assistants 114-1 to 114-N, or the automated medication dispensing system.

The detail functioning of the system 102 is described below with the help of figures.

The system may receive sensor data comprising at least one of motion data, occupancy data, and environmental data. It may be noted that each piece of sensor data is associated with a time stamp.

In one embodiment, the system 102 comprises a sensor network (110) configured to monitor a patient's living environment and collect a variety of sensor data in real-time. The sensor data includes motion data, occupancy data, and environmental data. In an embodiment, the system may add a time stamp to each piece of the sensor data to enable accurate temporal alignment across multiple sources. The system 102 leverages these time-stamped data points to continuously monitor the patient's activity levels, behaviors, and environmental conditions, providing dynamic insights into the patient's overall health status.

The motion data is collected via motion sensors strategically placed throughout key areas of the home, such as the living room, hallway, and bedroom. Motion sensors comprising one or more of a passive infrared sensor (PIR sensor), microwave sensors, tomographic sensors, ultrasonic sensors, camera-based sensors, millimeter-wave (mmWave) sensors, radar, LiDAR, gyroscope/accelerometer logs. These sensors detect movement within their respective ranges and help track the patient's mobility patterns and activity levels. Each detected motion event is tagged with a time stamp, ensuring that sequences of movement can be chronologically analyzed.

The occupancy data is derived from both door sensors and chair sensors. Door sensors are mounted on doors throughout the residence and record entry and exit times, capturing how often and when the patient moves between different rooms. The door sensors are nothing, but a Magnetic Contact Sensor mounted on doors and consist of a magnet and contact switch that triggers when the door opens or closes.

Chair sensors, integrated into seating areas, monitor the duration and frequency of sitting events, allowing for assessment of sedentary behavior or detection of mobility-related concerns. Both types of sensors provide data linked with time stamps, enabling the system to accurately correlate patient movements and behaviors over time. The chair sensors are nothing, but pressure sensors integrated into chairs configured to monitor sitting duration and frequency. Further, the bed mat sensors are nothing, but a Pressure or Capacitive Sensors configured to track sleep patterns, including sleep duration, restlessness, and optionally heart rate data.

The bed mat sensors, positioned beneath or within the patient's bed, collect sleep-related data such as sleep duration, restlessness, and, if integrated, heart rate. These readings are essential for evaluating sleep quality, which is a key indicator of the patient's well-being. As with other sensor types, the sleep data is time-stamped to ensure alignment with other activity patterns.

Environmental data is gathered through sensors that monitor parameters such as temperature, humidity, and light levels across various rooms in the living space. This contextual data complements the activity patterns by assessing environmental factors that may affect the patient's health, such as extreme temperatures or poor air quality. All environmental readings are captured with time stamps, providing a comprehensive and synchronized dataset that links the patient's activities to their surroundings.

Given the continuous and high-volume nature of sensor data—generated from multiple sensors, often at minute-level intervals—the system is designed for real-time data processing and efficient storage. Upon receipt, the sensor data is processed immediately, mapped, and synchronized across different sensor types. The system transforms this raw data into a standardized format, integrating room-specific information and time stamps, enabling efficient organization and alignment across all data streams. This real-time processing ensures that any significant changes in the patient's behavior or environment can be detected promptly. For example, if the system identifies prolonged inactivity coupled with environmental changes (e.g., a drop in room temperature), it can generate an alert to caregivers or medical professionals.

The system's efficient data storage architecture ensures that all sensor readings, along with their respective time stamps and contextual information, are securely stored in a structured database. Historical data is retained to establish baseline activity patterns, allowing for long-term tracking and comparison. These baselines help differentiate between normal behavioral variations and significant deviations that may indicate potential health risks.

To manage the massive influx of data and reduce the burden on human caregivers, the system employs automated algorithms for ongoing analysis. These algorithms continuously compare incoming real-time data against the patient's baseline activity patterns to identify anomalies or potential health risks. For instance, if the motion sensors detect no activity in the living room for an extended period while the environmental sensors report unusually low temperatures, the system correlates these data points and flags them as a potential concern, notifying caregivers with actionable insights.

By integrating and analyzing real-time sensor data—across motion, occupancy, environmental factors, and sleep patterns—the system provides a comprehensive and time-sensitive view of the patient's health and living conditions. This continuous monitoring capability supports early detection of risks, proactive intervention, and effective care management, ultimately enhancing patient safety and reducing the caregiver's burden.

In some implementations, the system may utilize millimeter wave sensing technology to perform gait analysis for assessing fall risk. Millimeter wave sensors may be installed in key areas of the living space to capture detailed data on a patient's walking patterns, speed, and stability. The high-resolution sensing capabilities of millimeter wave technology may enable detection of subtle changes in gait that could indicate increased fall risk.

The sensor network may collect raw data in various formats, such as binary signals, analog measurements, or time series data. This raw sensor data may then be processed and analyzed using advanced algorithms, including generative AI techniques.

Further to receiving the sensor data, the system enriches the collected sensor data by incorporating room-specific information. This process involves mapping the sensor data to specific areas within the living space, such as the bedroom, living room, or kitchen. By associating sensor data with particular rooms, the system can provide more detailed and contextual insights into the patient's activities and behaviors.

In some cases, the enrichment process may involve creating a digital map of the living space, with sensors assigned to specific locations. This allows for a more granular analysis of the patient's movements and activities within different areas of their home.

The enriched sensor data may be used to generate more accurate and meaningful activity patterns, as it provides context for the collected information. For example, prolonged inactivity detected in the bedroom may have different implications than similar inactivity detected in the living room.

By combining time-stamped sensor data with room-specific information, the system can create a comprehensive picture of the patient's daily routines, mobility patterns, and potential health-related behaviors. This enriched data serves as the foundation for further analysis and pattern recognition processes within the system.

The system may integrate various types of medical data associated with the patient to provide comprehensive health monitoring and analysis. This medical data may include information about the patient's medical conditions, treatment history, medication data, and biometric data.

In some cases, the medical conditions data may encompass a list of diagnosed conditions, chronic illnesses, or ongoing health concerns. This information may be used to contextualize the sensor data and activity patterns, allowing for more accurate anomaly detection and health risk prediction.

The treatment history data may include records of past medical procedures, hospitalizations, or ongoing therapies. In some implementations, this data may be used to track the patient's progress over time and identify potential correlations between treatments and changes in activity patterns.

Medication data may comprise a list of current prescriptions, dosage information, and medication schedules. The system may utilize this data to monitor medication adherence by correlating sensor data with expected medication intake times. In some cases, the system may generate reminders or alerts if deviations from the medication schedule are detected.

Vital sign data includes physiological health metrics such as heart rate, blood pressure, blood glucose levels, and other key indicators of the patient's physical condition. This data is collected through wearable devices (e.g., smartwatches, fitness trackers) or approved medical devices such as a blood pressure cuff, or a pulse oximeter, or glucometer, smart insulin dispensers integrated sensors (e.g., bed mat sensors with embedded heart rate monitors). The system analyzes this vital sign data in conjunction with activity patterns (such as mobility, sleep quality, and medication adherence) to generate a comprehensive assessment of the patient's health. By correlating physiological trends with behavioral patterns, the system enhances its ability to detect early signs of health deterioration and predict potential risks, supporting timely intervention.

In some implementations, the medical data may be securely imported from electronic health records (EHR) systems. The system may employ encryption and secure data transfer protocols, ensuring compliance with healthcare data regulations, such as HIPAA (Health Insurance Portability and Accountability Act) and HITECH (Health Information Technology for Economic and Clinical Health Act) in the United States, as well as GDPR (General Data Protection Regulation) in the European Union and other international standards. These protocols ensure the protection of PHI (Protected Health Information), PII (Personally Identifiable Information), and PCI (Payment Card Information) during storage, transmission, and processing, safeguarding patient privacy and promoting data security across all systems. The integration of medical data with sensor-derived activity patterns may enable the system to perform more sophisticated health risk assessments. For example, changes in activity patterns may be evaluated in the context of known medical conditions or recent treatments, potentially leading to earlier detection of health issues or more personalized care recommendations.

Further to the enrichment, the system may generate activity pattern data in real-time by processing enriched sensor data and medical data using a pattern recognition model. The pattern recognition model operates in two phases: first, it establishes baseline activity patterns by analyzing historical sensor data; second, it processes incoming real-time sensor data to generate current activity patterns. These patterns reflect various aspects of patient behavior and health, enabling subsequent comparison for anomaly detection. In some implementations, if the system receives a sufficient duration of real-time activity data that consistently aligns with the existing baseline and no anomalies are detected, the system may update the baseline activity pattern data accordingly. This adaptive mechanism ensures that the baseline remains accurate as the patient's behavior evolves over time.

The enriched sensor data may include raw motion data (e.g., gyroscope and accelerometer readings), occupancy data, and environmental data that has been augmented with room-specific information. This room-specific information provides spatial context for the sensor readings, allowing for accurate interpretation of patient activities in different areas of the living space.

Medical data processed alongside the enriched sensor data may include information about the patient's medical conditions, treatment history, medication data, and vital sign data (e.g., heart rate, blood pressure). By incorporating this medical context, the pattern recognition model generates comprehensive activity patterns that reflect both the patient's behaviors and health status.

The pattern recognition model employs various techniques to analyze the input data. In some cases, the model segments the sensor data into fixed-duration time-windowed blocks. For each time-windowed block, the model computes a feature vector containing statistical measures of the sensor data (e.g., mean, variance) and correlation measures between the sensor data and the medical data.

The model processes each feature vector using a neural network model, such as a convolutional neural network (CNN) for spatial pattern recognition or a recurrent neural network (RNN) for temporal pattern recognition. This process produces activity classifications, comprising class labels or probability scores corresponding to predefined categories related to the patient's health and activities (e.g., active, sedentary, asleep). These classifications are used for both establishing the baseline activity patterns and creating real-time activity patterns, which are later utilized in the anomaly detection model for comparative analysis.

The resulting activity pattern data may map the classification data to the corresponding time-windowed data blocks. This mapping may represent various behaviors of the patient, including:
1. Mobility: The activity pattern data may indicate the patient's movement patterns throughout the living space, including frequency and duration of movements between rooms.
2. Sleep patterns: By analyzing data from bed sensors and other relevant inputs, the algorithm may generate patterns related to sleep duration, quality, and consistency.
3. Medication adherence: The activity pattern data may reflect patterns associated with medication intake, potentially indicating whether the patient is following prescribed medication schedules.

In addition to these specific behaviors, the activity pattern data may include statistical measures of sensor data, temporal patterns, and correlations between sensor data and medical data. This comprehensive set of patterns may serve as a baseline for detecting anomalies and predicting potential health risks.

The real-time nature of this process allows for continuous updating of the activity pattern data as new sensor readings and medical information become available. This ongoing analysis may enable timely detection of changes in patient behavior or health status.

In an example, and not by way of limitation, the pattern recognition model analyzes real-time sensor data to identify health-related activity patterns. The system segments the incoming sensor data into fixed-duration time-windowed blocks (e.g., 30 minutes), processes each block to calculate feature vectors comprising statistical measures and correlation metrics between sensor data and medical data, and classifies these vectors using a neural network classifier. In some implementations, the classifier may be a Long Short-Term Memory (LSTM) neural network when temporal dependencies across time windows are relevant, or a Convolutional Neural Network (CNN) when analyzing spatial patterns within individual windows. The classification results are mapped to generate activity pattern data, reflecting patient behaviors such as mobility, sleep quality, and medication adherence.

The system receives raw sensor data in the form of log data, including continuous streams from motion sensors, door sensors, pressure sensors, bed mat sensors, and environmental sensors. This raw data may include readings from gyroscopes and accelerometers for motion sensors, pressure changes for seating sensors, and physiological signals such as heart rate from bed mat sensors. The system is configured to analyze this raw data in real-time, transforming it into meaningful insights that reflect patient activities and environmental conditions. This transformation involves segmenting the raw data into fixed 30-minute time windows, enabling consistent analysis across uniform periods without overwhelming computational resources.

TABLE 1
Example of Data Collected from Raw Sensor Logs

| Time | | |
|---|---|---|
| Window | Sensor Type | Data Collected (Derived from Raw Logs) |
| 00:00- | Motion sensors | Number of movement detections (from PIR |
| 00:30 | | motion sensors, microwave sensors, |
| | | tomographic sensors, ultrasonic sensors, |
| | | camera-based sensors, millimeter-wave |
| | | (mmWave) sensors, radar, LiDAR, |
| | | gyroscope/accelerometer logs) |
| | Door sensors | Number of door openings (log activations) |
| | Seating | Sitting duration (minutes, derived from |
| | pressure | pressure fluctuations) |
| | sensors | |
| | Bed mat | Sleep duration, restlessness index, heart |
| | sensors | rate (if integrated) |
| | Environmental | Average temperature, humidity (from |
| | sensors | environmental readings) |
| 00:30- | Same sensors | Next batch of sensor readings (same |
| 01:00 | | parameters) |

For each 30-minute time window, the system processes this log data to compute a feature vector, summarizing the transformed insights. This feature vector consolidates various statistical measures and correlation metrics derived from the raw data streams:

TABLE 2
Example Feature Vector Calculations

| Feature | Calculation | Data Source |
|---|---|---|
| Total movements | Sum of motion detections from | Motion sensors |
| | gyroscope/accelerometer logs | (processed from raw |
| | | logs) |
| Average sitting | Mean sitting time during the | Seating pressure |
| duration | window | sensors |
| Number of room | Count of door sensor activations | Door sensors |
| transitions | | |
| Sleep status | Binary indicator (1 = asleep, | Bed mat sensors |
| | 0 = awake) | |
| Restlessness | Variance in bed mat pressure | Bed mat sensors |
| index | readings | |
| Average heart | Mean heart rate (if integrated) | Bed mat sensors |
| rate | | |
| Average | Mean temperature during the | Environmental |
| temperature | time window | sensors |
| Motion- | Pearson correlation between | Motion + |
| environment | motion detections and | environmental |
| correlation | temperature fluctuations | sensors |
| Medication | Binary indicator (1 = dose taken | Medication |
| timing | within scheduled window, 0 = | records + seating |
| alignment | missed) | pressure sensor |
| | | (e.g., medicine |
| | | cabinet use) |

Each feature vector thus represents a structured set of numerical and categorical values, derived from raw sensor logs, summarizing the patient's physical activity, environmental conditions, and medication adherence for each time window. The system's ability to transform raw log data into actionable insights ensures real-time monitoring and efficient anomaly detection.

Further, the neural network classifier used in this example is a Long Short-Term Memory (LSTM) network, a type of Recurrent Neural Network (RNN) optimized for sequential data. The LSTM processes sequences of feature vectors across multiple time windows to capture temporal dependencies (e.g., gradual decline in mobility or disrupted sleep over time).
Classifier Inputs: Sequences of feature vectors from consecutive time windows.
Classifier Output:
   a. Class labels (e.g., Normal Activity, Low Mobility, Sleep Disturbance, Medication Non-adherence).
   b. Probability scores for each category (e.g., Low Mobility=0.85, Normal Activity=0.15).

### Classification Data Mapping

The classification data produced by the LSTM classifier is mapped back to the corresponding time-windowed data blocks. This activity pattern data represents the classified behaviors of the patient, forming a chronological map of health-related events and patterns.

### Illustrative Example

| Time | Classification | |
|---|---|---|
| Window | Label | Probability Scores |
| 00:00-00:30 | Normal Activity | Normal = 0.80, Low Mobility = 0.10, |
| | | Sleep Disturbance = 0.10 |
| 00:30-01:00 | Low Mobility | Normal = 0.20, Low Mobility = 0.75, |
| | | Sleep Disturbance = 0.05 |

This activity pattern data is continuously updated in real-time as new sensor data is received, allowing the system to maintain a dynamic baseline of the patient's behavior. Any deviation from this baseline—such as a sustained period of low mobility or a sleep disturbance—can trigger alerts for caregivers, guardians, or medical professionals, enabling timely interventions.

This process of segmentation, feature extraction, classification, and mapping ensures the system provides actionable insights based on comprehensive, contextual analysis of the patient's activity patterns and health status.

Further to generating the activity pattern data, the system may detect anomalies that indicate potential health risks by comparing current activity pattern data with baseline activity pattern data of the patient. In some cases, the baseline activity pattern data may be established over time as the system collects and analyzes sensor data for the patient.

The anomaly detection process may involve analyzing various aspects of the patient's activity patterns. For example, the system may compare current mobility patterns, sleep patterns, and medication adherence data to the established baseline for that patient. Deviations from the baseline that exceed predetermined thresholds may be flagged as potential anomalies.

In some cases, the system may employ statistical methods to identify anomalies. For instance, the system may calculate z-scores or other statistical measures to quantify how far current activity data deviates from the mean or median of the baseline data. Values that fall outside a specified range (e.g., more than two standard deviations from the mean) may be classified as anomalies.

The system may also utilize machine learning algorithms to detect anomalies. These algorithms may be trained on historical data to recognize patterns indicative of normal behavior for the patient. When current activity patterns deviate significantly from these learned patterns, the system may flag these deviations as potential anomalies.

In some implementations, the anomaly detection process may consider temporal aspects of the data. For example, the system may analyze the duration, frequency, and timing of activities compared to the patient's typical routines. Unusual changes in these temporal patterns may be identified as anomalies.

The system may also incorporate contextual information when detecting anomalies. For instance, changes in activity patterns may be evaluated in light of known medical conditions, recent treatments, or medication changes for the patient. This contextual analysis may help distinguish between benign variations and potentially concerning anomalies.

In some cases, the anomaly detection process may be adaptive, continuously updating the baseline as new data is collected. This approach may allow the system to account for gradual changes in the patient's behavior over time, while still detecting sudden or significant deviations that may indicate health risks.

The system may assign different weights or priorities to various types of anomalies based on their potential impact on the patient's health. For example, prolonged periods of inactivity or significant changes in sleep patterns may be given higher priority than minor variations in daily routines.

When an anomaly is detected, the system may generate an alert or notification. In some implementations, these alerts may be categorized based on severity or urgency, allowing caregivers or healthcare providers to prioritize their responses appropriately.

In an embodiment, a machine learning (ML) model is trained to detect anomalies in patient activity patterns. The ML model training is trained by utilizing a training dataset comprising historical sensor data, medical data, and previously detected anomalies. The training dataset includes labeled data representing both normal activity patterns and anomalous events that could indicate potential health risks, such as prolonged inactivity, disrupted sleep, or missed medication doses.

The training dataset is composed of:
Sensor Data: Time-stamped motion, occupancy, sleep, and environmental data collected from the patient's living environment.
Baseline Activity Patterns: Normal behavior patterns established over time (e.g., typical mobility and sleep routines).
Anomalies (Labeled Events): Historical deviations from baseline patterns (e.g., periods of inactivity longer than usual or unusual sleep disturbances).
Anomaly Weights (Health Risk Prioritization): Each anomaly is assigned a weight reflecting its potential health risk severity. For example:
Prolonged inactivity (>12 hours): High weight (e.g., 0.9)
Severe sleep disturbance (multiple awakenings/night): Moderate weight (e.g., 0.7)
Missed medication dose: High weight (e.g., 0.85)
Minor variations in movement frequency: Low weight (e.g., 0.3)

These weights guide the ML model during training to prioritize learning patterns associated with higher health risks.

### Training Process:

### A) Feature Extraction:

For each time-windowed data block, the system computes feature vectors comprising statistical measures (e.g., mean, variance), temporal patterns, and correlation metrics between sensor data and medical records.

### Example Features:

Average sitting duration per hour
Number of room transitions per day
Variance in heart rate overnight
Deviation from scheduled medication time

### B) Labeling and Weight Assignment:

Normal patterns and anomalous events are labeled in the dataset.

Anomaly weights are assigned to each labeled event based on the potential impact on the patient's health (as described above).

### C) Model Selection:

The system uses a Recurrent Neural Network (RNN) with Long Short-Term Memory (LSTM) layers to capture temporal dependencies in the time-series activity data.

The LSTM processes sequences of feature vectors to learn both short-term fluctuations and long-term behavioral trends.

D) Training with Weighted Anomalies:

During training, the anomaly weights are incorporated into the loss function (e.g., weighted cross-entropy loss), ensuring that the model gives more importance to critical anomalies (such as prolonged inactivity) compared to less significant deviations (like minor movement variations).

The model is optimized to minimize weighted classification errors, ensuring higher sensitivity for high-priority anomalies.

### E) Validation and Calibration:

The model's accuracy is validated using a separate validation set, which also includes weighted anomalies.

Probability thresholds are calibrated to ensure appropriate sensitivity and specificity for different types of anomalies.

### Example Table: Weighted Anomalies in Training Dataset

| | | Anomaly | |
|---|---|---|---|
| Event Type | Anomaly Detected | Weight | Feature Trigger |
| Normal | Routine movement | 0 | Standard motion and |
| activity | patterns | | occupancy data |
| Prolonged | No motion detected | 0.9 | Low motion count, |
| inactivity | for 12+ hours | | high sitting duration |
| Sleep | Multiple awakenings | 0.7 | High restlessness |
| disturbance | per night | | index, interrupted |
| | | | sleep |
| Missed | No activity detected | 0.85 | No door sensor |
| medication | near med cabinet | | trigger near cabinet |
| dose | | | |
| Minor | Slightly fewer room | 0.3 | Reduced door sensor |
| routine | transitions | | activations |
| variation | | | |

### Anomaly Detection in Practice (Post-Training):

Once deployed, the trained ML model processes real-time feature vectors.

For each time window, the model outputs probability scores for predefined anomaly categories.

If an anomaly is detected, the system references the learned anomaly weights to prioritize alerts (e.g., high-risk anomalies trigger immediate notifications).

For example, if the model detects prolonged inactivity with a high probability (e.g., 0.95), given its weight (0.9), the system triggers a high-priority alert to caregivers.

In some cases, the system may utilize a health prediction model to assess the patient's health based on detected anomalies and activity pattern data. The health prediction model may be a machine learning algorithm trained on data from multiple patients and medical records to recognize patterns correlated with various health conditions.

The health prediction model may be implemented as a neural network, such as a deep learning model. In some implementations, the model may be trained using supervised learning techniques on a large dataset comprising historical sensor data and corresponding medical outcomes from a diverse population of patients. This training data may include activity patterns, detected anomalies, and associated health conditions or events.

The input features for the health prediction model may include:
1. Activity pattern data derived from processed sensor data
2. Detected anomalies in the patient's behavior or routines
3. Statistical measures of sensor data
4. Temporal patterns in the patient's activities
5. Correlations between sensor data and medical data

In some cases, the health prediction model may also incorporate the patient's specific medical history, demographic information, and known risk factors as additional input features. This personalization may allow the model to make more accurate predictions tailored to the individual patient.

The health prediction model may output probabilities or risk scores for various health conditions or events. For example, the model may predict the likelihood of falls, cognitive decline, or exacerbation of chronic conditions based on the input data.

To improve its performance over time, the health prediction model may employ continuous learning techniques. As new data becomes available from the monitored patient and other patients in the system, the model may be periodically retrained or fine-tuned to incorporate this new information.

In some implementations, the health prediction model may use ensemble methods, combining predictions from multiple sub-models to improve overall accuracy and robustness. These sub-models may include different types of algorithms, such as decision trees, support vector machines, and recurrent neural networks, each potentially capturing different aspects of the patient's health status.

The health prediction model may also incorporate temporal aspects of the data, recognizing that changes in activity patterns over time may be indicative of evolving health conditions. In some cases, the model may use techniques such as time series analysis or recurrent neural networks to capture these temporal dependencies.

To enhance interpretability, the health prediction model may provide feature importance scores, indicating which input features contributed most significantly to a particular prediction. This information may be valuable for healthcare providers in understanding the basis for the model's predictions and in formulating appropriate interventions.

In some implementations, the health prediction model may be designed to handle missing or noisy data, which may occur due to sensor malfunctions or temporary changes in the patient's environment. Techniques such as data imputation or robust feature engineering may be employed to ensure the model can still make reliable predictions in these scenarios.

The health prediction model may be subject to regular validation and performance monitoring to ensure its predictions remain accurate and clinically relevant. This process may involve comparing the model's predictions against actual health outcomes and adjusting the model as necessary to maintain its predictive power.

In one implementation, the system employs a health prediction model designed to assess the patient's risk of health conditions based on detected anomalies and activity pattern data. This model is implemented as a deep neural network (DNN), specifically a Recurrent Neural Network (RNN) with Long Short-Term Memory (LSTM) layers, which are well-suited for capturing temporal dependencies in sequential activity data.

The health prediction model is trained using supervised learning techniques on a large-scale dataset that includes historical sensor data, detected anomalies, and corresponding medical outcomes from a diverse population of patients. The training dataset spans multiple health scenarios, including mobility decline, fall incidents, sleep disturbances, and medication non-adherence, allowing the model to generalize across various conditions.

### Input Features for the Health Prediction Model:

The model uses a rich set of input features to make personalized health predictions:

| Input Feature | Description |
|---|---|
| Activity Pattern Data | Processed from sensor inputs (motion, |
| | occupancy, sleep, environmental data). |
| Detected Anomalies | Deviations from baseline patterns (e.g., |
| | prolonged inactivity, sleep disruptions). |
| Statistical Measures of | Metrics like mean, variance, and frequency |
| Sensor Data | from different sensor modalities. |
| Temporal Patterns | Sequence-based trends in patient activities |
| | (e.g., daily mobility fluctuations). |
| Correlations Between | Cross-references between activity levels and |
| Sensor and Medical Data | medical conditions or treatments. |
| Patient Medical History, | Includes age, chronic conditions, medication |
| Demographics, Risk | schedules, and other health data. |
| Factors | |

### Model Outputs:

The health prediction model generates risk scores (probability values) for potential health conditions. For instance:

| Health Event | Predicted Risk Score (Probability) |
|---|---|
| Fall Risk within next 7 days | 0.78 |
| Cognitive Decline Indicators | 0.65 |
| Sleep Disorder Aggravation | 0.83 |
| Medication Non-Adherence | 0.92 |

These risk scores provide quantifiable assessments of the likelihood that the patient will experience specific health events based on recent activity patterns and anomalies.

### Model Training Example:

A) Dataset Size: 10,000 patient profiles with 12 months of activity data and health outcomes.
B) Training Data Composition:
Normal behavior sequences labeled as “healthy.”
Anomalous events (e.g., prolonged inactivity, missed medication) labeled with specific health outcomes (e.g., falls, hospitalization).
C) Weighted Anomalies: Anomalies in the training set are weighted by severity (e.g., fall-related anomalies have higher weights than minor routine deviations). These weights influence the loss function, ensuring the model prioritizes critical health risks during training.

### Ensemble Learning Approach:

To enhance predictive performance, the system may employ an ensemble method, combining predictions from multiple sub-models:

| Sub-Model Type | Role |
|---|---|
| Decision Tree Classifier | Handles structured tabular data. |
| Support Vector Machine (SVM) | Captures non-linear patterns in static |
| | features. |
| LSTM-based Neural Network | Captures temporal dependencies in |
| | sequential data. |

The final prediction is made by aggregating the outputs (e.g., through weighted averaging) from these sub-models, improving overall accuracy and robustness.

### Adaptive Learning and Continuous Improvement:

The health prediction model supports continuous learning. As new data becomes available from the monitored patient and other users, the model is retrained periodically to incorporate recent trends. For example, if a patient's mobility declines gradually, the model adapts its baseline and recalibrates the risk scores accordingly.

### Handling Noisy or Missing Data:

The model includes mechanisms for robust data handling, such as data imputation for filling missing values and feature engineering techniques that ensure reliable predictions despite potential sensor malfunctions or gaps in data collection.

### Interpretability and Clinical Relevance:

To aid healthcare providers, the model outputs feature importance scores alongside predictions. These scores highlight which input factors (e.g., mobility reduction, sleep disturbances) contributed most to a specific risk assessment, helping clinicians understand the basis for the model's decisions and enabling more informed interventions.

### Model Monitoring and Validation:

The system regularly validates the health prediction model by comparing predicted risk scores against actual patient outcomes (e.g., reported falls, confirmed medication adherence). This ensures the model maintains high accuracy and remains clinically relevant, with periodic adjustments as necessary.

As new data is collected and processed by the system, a health prediction engine may continuously update and refine its models. This ongoing learning process may allow the system to improve its predictive accuracy over time and adapt to changes in individual patient behaviors or health status. In some cases, the module may incorporate feedback from caregivers or annotations from medical professionals to further enhance its predictive capabilities.

The health prediction engine may be capable of identifying a wide range of potential health risks. For example:
1. Fall risk: By analyzing changes in gait patterns, mobility data, and medication information, the module may predict an increased likelihood of falls.
2. Cardiovascular issues: The system may detect subtle changes in heart rate patterns or blood pressure readings that could indicate an elevated risk of cardiac events.
Respiratory Diseases: Subtle changes in the respiratory rate patterns that could indicate elevated risk of COPD, Emphysema, Asthma
3. Cognitive decline: Changes in daily routines, medication adherence, or social interaction patterns may be used to predict potential cognitive impairment.
4. Urinary tract infections: The module may identify increased nighttime bathroom visits or changes in hydration patterns that could signal a developing UTI.
5. Depression or anxiety: Analysis of sleep patterns, activity levels, and social interactions may help predict the onset or worsening of mental health conditions.

Based on these predictions, the health prediction engine may generate alerts and reports for caregivers or medical professionals. In some implementations, the system may categorize alerts based on urgency and potential impact on patient health. For example, a high-risk fall prediction may trigger an immediate alert, while a gradual increase in cognitive decline risk may be included in a weekly summary report.

The health prediction engine may also be used to generate insurance risk scores and inform premium calculations. In some cases, the system may provide aggregated, anonymized data to insurance providers to help them assess population-level health risks and trends. This information may be used to develop more personalized insurance products or to incentivize preventive care measures.

By leveraging advanced predictive algorithms and continuous learning capabilities, the health prediction engine may enable more proactive and personalized patient care strategies. The module's ability to identify potential health risks before they manifest as serious issues may support early intervention and improved health outcomes.

In an embodiment, the prediction model in the patient care system is designed to forecast potential health risks such as falls, sleep disturbances, or medication non-adherence. This model is implemented as a deep neural network (DNN), specifically a Recurrent Neural Network (RNN) with Long Short-Term Memory (LSTM) layers. The LSTM architecture is well-suited for handling time-series sensor data and capturing temporal dependencies across multiple patient activities and health indicators. The model takes as input a combination of activity pattern data, detected anomalies, temporal trends, and medical history, allowing it to make personalized predictions based on both recent and long-term behavior patterns.

The prediction model is trained using supervised learning techniques on a large historical dataset collected from multiple patients. This dataset includes sensor data sequences, detected anomalies, patient outcomes, and medical records. Input features are extracted through feature engineering, including statistical measures (mean, variance), temporal patterns (daily mobility trends, sleep disruptions), and correlations between sensor readings and medical events (e.g., medication adherence). The output labels consist of binary health event flags (e.g., fall/no fall) or risk scores (probability of an event occurring). The model is trained to minimize a loss function (e.g., cross-entropy for classification or mean squared error for regression), optimized using algorithms like Adam. The training process incorporates regularization techniques (e.g., dropout layers) to prevent overfitting, ensuring robust generalization across diverse patient populations.

The neural network architecture includes multiple hidden layers, each designed to process sequential data and extract meaningful patterns. The LSTM layers form the core of the architecture, capable of retaining long-term dependencies and filtering irrelevant information through gates (input, output, and forget gates). The LSTM layers are followed by dense (fully connected) layers, which further refine the extracted features and generate risk scores or probability outputs. For instance, the model may include two LSTM layers (with 64 and 32 units) followed by dense layers (with 64 and 32 neurons), activated using ReLU functions for non-linearity. The output layer uses a sigmoid function for binary risk predictions (e.g., risk of fall=0.78). The model operates in real-time, processing incoming activity pattern data and anomalies, continuously updating risk assessments to provide proactive alerts to caregivers or medical professionals.

Further to predicting the health of the patient, the system may generate patient care instructions based on the predicted health of the patient. These instructions may be tailored to address specific health concerns or anomalies detected through the analysis of sensor data and activity patterns.

In some cases, the patient care instructions may include client case overviews for newer caregivers. These overviews may provide a summary of the patient's medical history, current health status, and any specific care requirements. For example, the instructions may include information about the patient's mobility limitations, dietary restrictions, or cognitive impairments.

The system may also generate instructions related to medication changes or dosage adjustments. In some cases, if the analysis of sensor data indicates a potential issue with medication adherence, the instructions may include reminders or suggestions for improving compliance. For instance, the instructions may recommend setting up specific times for medication administration or using pill organizers to help the patient stay on track.

Daily routines may be incorporated into the patient care instructions as well. The system may analyze the patient's activity patterns and generate recommendations for structuring the day to promote better health outcomes. These instructions may include suggestions for meal times, exercise periods, and rest intervals tailored to the patient's specific needs and capabilities.

In some cases, the patient care instructions may address specific anomalies detected by the system. For example, if the sensor data indicates a change in the patient's sleep patterns, the instructions may include recommendations for improving sleep hygiene or adjusting the bedroom environment.

The system may also generate instructions related to mobility and fall prevention based on the analysis of motion sensor data. These instructions may include recommendations for assistive devices, environmental modifications, or specific exercises to improve balance and strength.

In some cases, the patient care instructions may be updated in real-time based on ongoing analysis of sensor data and health predictions. This may allow for rapid response to changing health conditions or emerging concerns.

The generated instructions may be delivered through various channels, such as a caregiver mobile application, a patient interface device, or an automated medication dispensing system. This flexibility in delivery methods may help ensure that the appropriate caregivers or healthcare providers receive timely and relevant information to support patient care.

The system may utilize secure communication channels to deliver patient care instructions to various interfaces and systems. These channels may employ encryption protocols such as TLS (Transport Layer Security), SSL (Secure Sockets Layer), or AES (Advanced Encryption Standard) to ensure the protection of sensitive patient information during transmission. The patient care instructions may be delivered to patient interface devices, including smartphones, tablets, specialized medical devices, conversational voice-activated devices (e.g., Alexa), or smart glasses and smart hearing aids. These devices may receive instructions through secure mobile applications or encrypted messaging systems that comply with healthcare data regulations, including HIPAA (Health Insurance Portability and Accountability Act) and HITECH (Health Information Technology for Economic and Clinical Health Act) in the United States, as well as GDPR (General Data Protection Regulation) in the European Union, ensuring that all transmitted data adheres to the highest standards for protecting PHI (Protected Health Information) and PII (Personally Identifiable Information).

In some cases, caregiver mobile applications may be used to receive and display patient care instructions. These applications may require secure authentication methods, such as multi-factor authentication, to ensure that only authorized caregivers can access the instructions. The caregiver mobile applications may provide real-time updates and notifications regarding patient care instructions, allowing for timely and efficient care delivery.

Automated medication dispensing systems may also receive patient care instructions through secure communication channels. These systems may be programmed to dispense medications according to the received instructions, helping to ensure accurate medication adherence. The communication between the central system and the automated medication dispensing systems may be secured using protocols such as HTTPS or VPN connections.

In some implementations, voice assistants such as Alexa™ may be utilized to deliver caregiver instructions. This method may provide an auditory interface for caregivers to access important information hands-free. To maintain security and privacy, a secure password distribution method may be employed for caregivers to access instructions through voice assistants.

The secure password distribution method may involve multiple steps to ensure that only authorized caregivers can access patient care instructions. In some cases, a home care manager may securely distribute passwords to caregivers through physical handover or secure electronic means such as encrypted emails or text messages. The system may generate unique passwords for each client, which caregivers must use to authenticate their access to instructions for specific patients.

To further enhance security, the voice assistant may require caregivers to provide the correct password before accessing any patient-specific information or instructions. This authentication process may help prevent unauthorized access to sensitive patient data while allowing legitimate caregivers to quickly and easily obtain the information they need to provide effective care.

In some cases, the system may generate natural language summaries of patient data using a fine-tuned Large Language Model (LLM). The LLM may be trained on historical sensor data and medical data to produce actionable insights related to patient care.

The process of creating natural language summaries may involve several steps. First, the system may process the activity pattern data generated from the sensor data and medical data. This processed data may serve as input for the LLM. The LLM may then analyze this input and generate a human-readable summary that describes the patient's current status, any detected anomalies, and potential health risks.

In some cases, the natural language summaries may include information about the patient's mobility patterns, sleep quality, medication adherence, and other relevant health indicators. The summaries may also highlight any significant changes or trends in the patient's behavior or health status.

The system may use these natural language summaries to generate patient care instructions. These instructions may specify one or more actions to be performed to assist the patient. For example, if the summary indicates a decline in the patient's mobility, the system may generate instructions for caregivers to encourage more physical activity or to assist with specific exercises.

In some cases, the patient care instructions may include recommendations based on the insights provided in the natural language summary. These recommendations may include situation advice for the patient, suggestions for modifying a treatment plan, medication reminders, or alerts for medical assistance when necessary.

The use of a fine-tuned LLM for generating natural language summaries may offer several advantages. It may allow for more nuanced and context-aware interpretations of the patient data, potentially leading to more personalized and effective care instructions. Additionally, the natural language format may make the information more accessible and understandable for caregivers and family members who may not have specialized medical knowledge.

In another embodiment, the system may employ Generative AI techniques to convert raw sensor data into natural language descriptions of patient behavior and health status. In some cases, machine learning models may be trained on large datasets of sensor readings and corresponding behavioral observations. These models may then generate human-readable summaries of patient activities, sleep patterns, medication adherence, and other relevant health indicators. For example, the system may produce a report stating “The patient has been less active than usual over the past week and has shown irregular sleep patterns, potentially indicating a decline in overall health status.”

The data acquisition and processing unit may also incorporate external data sources to provide additional context for patient care recommendations. In some implementations, local weather data may be integrated into the system's analysis. This weather information may be used to generate context-aware instructions for caregivers and patients. For example, on a day with extreme heat, the system may advise increased fluid intake and limited outdoor activity for patients with certain health conditions.

The instruction generation process may begin by categorizing the detected patterns or anomalies into different health domains, such as mobility, medication adherence, sleep quality, or vital signs. For each domain, the system may have a set of predefined instruction templates that can be customized based on the specific patient data and context.

In some implementations, the system may integrate with specialized healthcare language models, such as Hippocratic AI™, to generate more nuanced and medically appropriate instructions. These models may be trained on vast corpora of medical literature and clinical guidelines, allowing them to produce contextually relevant and accurate healthcare recommendations.

The system may employ a multi-level approach to determine the urgency and appropriate response for each detected issue. In some cases, the system may utilize a scoring mechanism that assigns numerical values to various factors, including:
1. Severity of the detected anomaly
2. Patient's medical history and risk factors
3. Frequency and duration of the observed pattern
4. Potential impact on overall health status

Based on the calculated scores, the system may categorize alerts into low, medium, and high-severity levels. Each severity level may have predefined thresholds and corresponding actions:
1. Low-severity alerts (e.g., score<30): The system may generate general wellness advice or minor lifestyle adjustments. These instructions may be delivered directly to the patient or caregiver without immediate medical professional involvement.
2. Medium-severity alerts (e.g., score 30-70): The system may provide more specific care instructions and recommend closer monitoring. In some cases, these alerts may be flagged for review by a nurse or care coordinator within a certain timeframe.
3. High-severity alerts (e.g., score>70): The system may immediately escalate the issue to a medical professional for review and potential intervention. These alerts may trigger urgent notifications to the patient's healthcare team.

The system may also incorporate machine learning algorithms to dynamically adjust its decision-making process based on feedback and outcomes. In some implementations, the system may learn from the actions taken by healthcare professionals in response to previous alerts, refining its ability to distinguish between issues that require immediate medical attention and those that can be addressed through automated instructions.

For example, if the system detects a sudden increase in nighttime bathroom visits coupled with changes in vital signs, it may initially categorize this as a medium-severity alert. The generated instructions may include recommendations for fluid intake management and suggest monitoring for potential urinary tract infection symptoms. However, if the patient's medical history includes recurrent UTIs or other risk factors, the system may elevate the alert to high-severity, prompting immediate review by a healthcare provider.

In some cases, the system may also consider the cumulative effect of multiple low or medium-severity alerts over time. If a pattern of minor issues persists or worsens despite the provided instructions, the system may escalate the overall situation to a higher severity level, ensuring that subtle but potentially significant health changes are not overlooked.

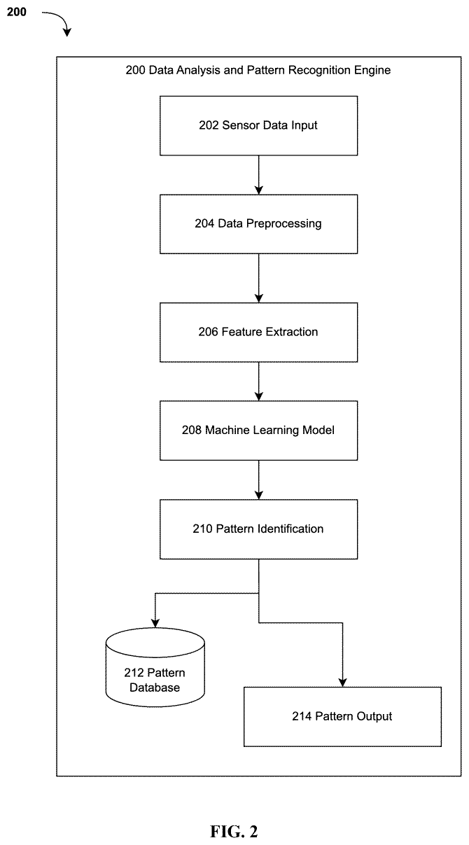

Referring to FIG. 2, a data analysis and pattern recognition engine 200 may process and analyze sensor data to identify patterns related to patient health and behavior. In some cases, the data analysis engine 200 may be implemented as software instructions executed by the processor 106 of the computing server 102.

The data analysis engine 200 may begin with sensor data input 202, which receives data collected by the first sensor 110-1, the second sensor 110-2, and the nth sensor 110-N. This sensor data may include health parameters, activity data, and medication adherence information associated with a patient.

From the sensor data input 202, the process may continue to data preprocessing 204. In some implementations, the data preprocessing 204 may involve cleaning the raw sensor data, handling missing values, and normalizing data from different sensor types.

Following data preprocessing 204, the data analysis engine 200 may perform feature extraction 206. The feature extraction 206 may identify relevant characteristics or patterns within the preprocessed sensor data. In some cases, this step may involve calculating statistical measures, identifying temporal patterns, or deriving higher-level features from the raw sensor inputs.

The extracted features may then be provided to a machine learning model 208. In some implementations, the machine learning model 208 may utilize various algorithms such as neural networks, decision trees, or support vector machines to analyze the extracted features. The machine learning model 208 may be trained on historical patient data to recognize patterns associated with different health conditions or behaviors.

Based on the analysis performed by the machine learning model 208, the data analysis engine 200 may proceed to pattern identification 210. The pattern identification 210 may extract one or more patterns from the processed sensor data. In some cases, these patterns may include:
1. Patterns related to health parameters, such as trends in heart rate, blood pressure, or respiratory rate
2. Patterns related to activity data, including daily routines, mobility levels, or sleep cycles
3. Patterns associated with medication adherence data, such as consistency in taking prescribed medications
4. Patterns related to medical data of the patient, which may incorporate historical health information

The identified patterns may be stored in a pattern database 212 for future reference and analysis. Additionally, the data analysis engine 200 may generate a pattern output 214, which may provide insights and summaries based on the identified patterns.

In some implementations, the data analysis engine 200 may employ generative AI techniques to convert the raw sensor data and identified patterns into natural language text. This natural language output may be used for documentation purposes, providing human-readable summaries of patient behaviors and health status. For example, the system may generate a report stating that—“The patient's activity levels have decreased by 20% over the past week, and nighttime restlessness has increased.”

By utilizing machine learning algorithms and advanced analytical techniques, the data analysis and pattern recognition engine 200 may enable comprehensive analysis of patient data, supporting more informed and personalized care decisions.

The health prediction engine may be implemented as a method 300 for predicting patient health status based on sensor data analysis. In some cases, the method 300 may be executed by the processor 106 of the computing server 102.

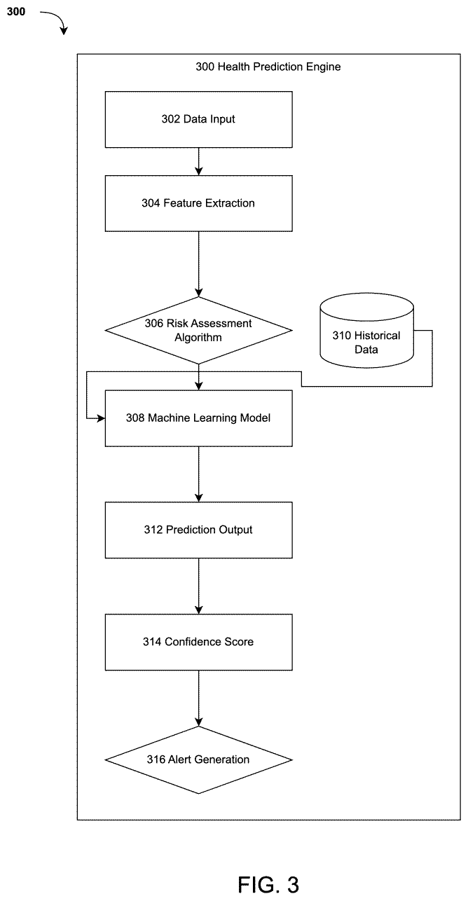

Referring to FIG. 3, a method 300 may begin with a data input step 302, where sensor data associated with a patient is received. This sensor data may be collected from the first sensor 110-1, the second sensor 110-2, and the nth sensor 110-N placed in the patient's residence. The sensor data may include health parameters, activity data, and medication adherence information.

Following the data input step 302, the method 300 may proceed to a feature extraction step 304. In some implementations, the feature extraction step 304 may involve identifying relevant characteristics or patterns within the preprocessed sensor data. This step may utilize techniques similar to those employed in the feature extraction 206 of the data analysis engine 200.

The method 300 may then continue to a risk assessment algorithm step 306. In some cases, the risk assessment algorithm step 306 may analyze the extracted features to identify potential health risks. The risk assessment algorithm step 306 may incorporate various factors to assess patient health status, including medication information, bowel movement patterns, and incontinence data.

A historical database 310 may provide input to both the risk assessment algorithm step 306 and a machine learning model step 308. The historical database 310 may contain past patient data, allowing the system to compare current patterns with historical trends.

The machine learning model step 308 may process the data from the risk assessment algorithm step 306 and the historical database 310. In some implementations, the machine learning model step 308 may employ predictive algorithms to identify potential health risks based on the extracted patterns.

Following the machine learning model step 308, the method 300 may proceed to a prediction output step 312. The prediction output step 312 may generate health predictions based on the analysis performed in the previous steps.

The method 300 may include a confidence score step 314, which evaluates the reliability of the predictions generated in the prediction output step 312. In some cases, the confidence score may be based on factors such as the quality and quantity of available data, the consistency of observed patterns, and the historical accuracy of predictions for similar cases.

The method 300 may conclude with an alert generation step 316. In some implementations, the alert generation step 316 may determine whether alerts should be generated based on the predictions and confidence scores. For example, if a high fall risk is predicted with a high confidence score, the system may generate an urgent alert for caregivers.

In some cases, the method 300 may incorporate millimeter wave sensing or radar technology to perform gait analysis and pace analysis of the patient for fall risk assessment or collect and transmit data from assistive mobility devices such as smart walkers, or smart walking sticks (canes) or similar assistive devices. This technology may be integrated into the sensor network, providing detailed data on the patient's walking patterns and stability. The gait analysis or pace analysis data may be processed in the feature extraction step 304 and incorporated into the risk assessment algorithm step 306.

The method 300 may generate a composite fall risk score based on multiple factors. In some implementations, this score may consider elements such as:
1. Gait analysis data from millimeter wave or radar sensors
2. Medication information, including types and dosages of prescribed drugs
3. Frequency and timing of bathroom visits, indicating potential incontinence issues
4. Historical fall incidents
5. Overall mobility patterns detected by motion sensors
6. Sleep quality and nighttime activity

The composite fall risk score may be calculated in the risk assessment algorithm step 306 and refined in the machine learning model step 308. In some cases, the score may be updated in real-time as new sensor data is received, allowing for continuous monitoring of fall risk.

By integrating multiple data sources and employing advanced analytical techniques, the health prediction engine implemented through the method 300 may provide comprehensive and personalized health risk assessments. This approach may enable more proactive and targeted interventions to maintain patient health and safety.

Although the system is described with reference to a pattern recognition engine, anomaly detection engine, and health prediction engine, each engine represents a set of instructions executed by a processor to perform specific computational tasks. These engines are not standalone physical components but are functional entities implemented through software routines executed by the processor. In one embodiment, the processor is configured to execute machine learning models and data processing algorithms associated with these engines, including feature extraction, pattern generation, anomaly detection, and risk prediction. The pattern recognition engine and health prediction engine refer to the logical sequence of operations, such as segmenting sensor data, computing feature vectors, and executing neural network models, all performed by the processor. Accordingly, the engines and the processor may be considered functionally analogous, where the processor provides the hardware execution environment, and the engines represent the adaptive, data-driven operations performed by the processor.

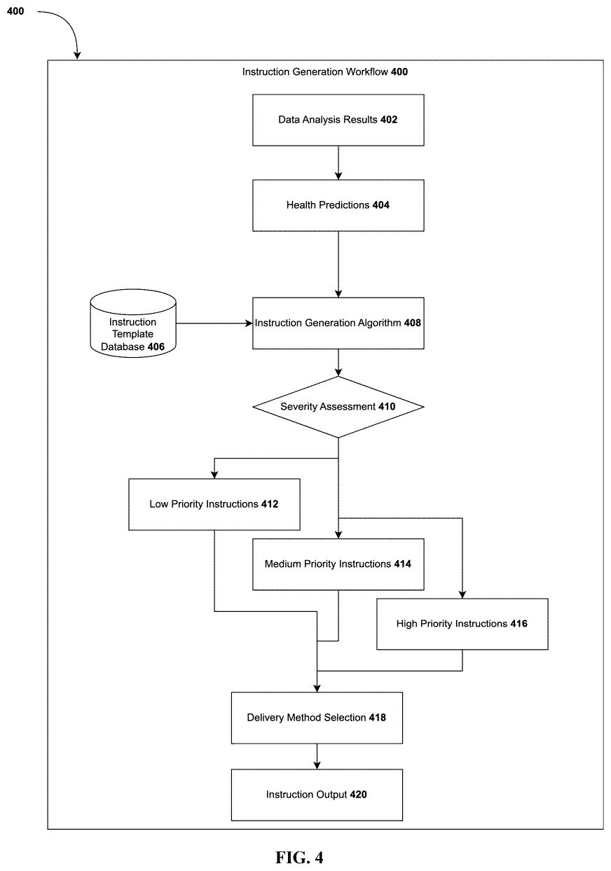

Referring to FIG. 4, an instruction generation workflow 400 may process data analysis results and health predictions to generate patient care instructions. In some cases, the workflow 400 may be implemented as software instructions executed by the processor 106 of the computing server 102.

The workflow 400 may begin with data analysis results 402, which provide input data for the process. This input may include patterns and anomalies identified by the data analysis engine 200. The workflow 400 may then proceed to health predictions 404, where the analyzed data is used to make predictions about patient health status.

An instruction template database 406 may provide input to an instruction generation algorithm 408. In some implementations, the instruction template database 406 may contain pre-defined templates for various health conditions and care scenarios. The instruction generation algorithm 408 may process the health predictions 404 along with templates from the instruction template database 406 to create tailored care instructions.

In some cases, the instruction generation algorithm 408 may utilize a Large Language Model (LLM) or Generative AI to generate the patient care instructions. This approach may allow for more nuanced and context-aware instruction generation based on the specific patient data and health predictions.

Following the instruction generation algorithm 408, the workflow 400 may move to a severity assessment 410. The severity assessment 410 may evaluate the urgency and potential impact of the identified health issues. Based on the severity assessment 410, the workflow 400 may branch into three possible paths: low priority instructions 412, medium priority instructions 414, or high priority instructions 416.

After the priority-specific instructions are generated, the workflow 400 may proceed to delivery method selection 418. The delivery method selection 418 may determine how the instructions will be conveyed. In some cases, this step may consider factors such as the urgency of the instructions, the preferred communication methods of the recipients, and the availability of different delivery channels.

The workflow 400 may conclude with instruction output 420, where the generated instructions are provided through the selected delivery method. In some implementations, the generated patient care instructions may be delivered to at least one of the patient, a guardian of the patient, a caregiver, and a remote care coordinator associated with the patient via a personal digital assistant. The personal digital assistant may include devices such as the first digital assistant 114-1, the second digital assistant 114-2, or the nth digital assistant 114-N.

The instruction generation module may be configured to generate at least one of situational advice, a new care plan, modification to an existing care plan, and recommendations for wellbeing of the patient based on the identified potential health risks. For example, if the health predictions 404 indicate an increased fall risk, the generated instructions may include recommendations for home safety modifications and suggestions for balance exercises.

In some cases, the system may detect weather conditions of the patient's residence and recommend changes to create an optimal living environment. For instance, if extreme heat is detected, the instructions may include advice on staying hydrated and using air conditioning.

The workflow 400 may also incorporate an Electronic Visit Verification (EVV) process for caregivers. The EVV may include authenticating the caregiver, storing caregiver profile data, storing the date and time of instruction delivery, and storing the duration of the caregiver visit. This process may help ensure the security of patient information and maintain accurate records of care provision.

Additionally, the instruction generation workflow 400 may facilitate the delivery of educational and entertainment content to the patient via the personal digital assistant. This content may be tailored based on the patient's health status, cognitive function, and personal preferences as determined by the data analysis and health prediction processes.

By integrating multiple data sources, employing advanced language generation techniques, and incorporating a severity-based triage system, the instruction generation workflow 400 may provide comprehensive and personalized care guidance while efficiently managing healthcare resources.

Exemplary embodiments discussed above may provide certain advantages. Though not required to practice aspects of the disclosure, these advantages may include those provided by the following features.

The system improves data processing efficiency by synchronizing diverse sensor data streams (motion, occupancy, environmental) in real-time, ensuring temporal alignment without manual intervention. This allows for accurate correlation of multi-modal data and improves the computational handling of time-series data, enhancing the overall reliability of health monitoring systems.

The system integrates an automated anomaly detection model that continuously compares real-time activity data against a baseline, minimizing the need for manual monitoring by caregivers. This reduces human resource dependency and enables the computer system to autonomously flag potential health risks, optimizing the efficiency of the monitoring infrastructure.

The system employs adaptive learning to continuously update baseline activity patterns as new sensor data is received, accounting for gradual behavior changes in the patient. This prevents the need for frequent manual recalibration, improving system accuracy over time and enhancing computational resource utilization by avoiding redundant recalculations.

The use of weighted anomaly inputs during machine learning training allows the system to prioritize high-severity health risks, enhancing model efficiency by focusing computational resources on the most critical predictions. This approach improves the accuracy and responsiveness of the prediction model, directly contributing to improved technical performance in the health monitoring domain.

The system incorporates data imputation techniques and robust feature engineering to handle missing or noisy sensor data, improving model reliability and computational efficiency. This ensures that even in scenarios where sensor malfunctions occur, the system can continue processing data without significant performance degradation, enhancing the technical robustness of patient monitoring solutions.

A number of implementations have been described. Nevertheless, it will be understood that various modifications may be made without departing from the spirit and scope of the disclosure. Accordingly, other implementations are within the scope of the following claims.

1. A method comprising:
receiving, by a processor, in real-time from a sensor network, sensor data comprising at least one of motion data, occupancy data, and environmental data, wherein each piece of sensor data is associated with a time stamp;
enriching, by the processor, the sensor data by incorporating room-specific information;
generating, by the processor, in real time, activity pattern data by processing the enriched sensor data and medical data of the patient by using a pattern recognition model, wherein the activity pattern data comprises the activity data of the patient being indicative at least one of mobility, sleep patterns, and medication adherence, statistical measures of sensor data, temporal patterns, and correlation between the sensor data and the medical data;
detecting, by the processor, an anomaly indicating a potential health risk, wherein the anomaly is detected by comparing the activity pattern data with a baseline activity pattern data of the patient;
predicting, by the processor, health of the patient based on the anomaly and the activity pattern data by utilizing a prediction model, wherein the prediction model is trained on data from other patients, medical data associated with the patient, to recognize patterns that correlate with health conditions;
generating, by the processor, patient care instructions based on the predicted health of the patient; and
delivering, by the processor, the patient care instructions through a secure communication channel to at least one of a patient interface device, a caregiver mobile application, or an automated medication dispensing system.

2. The method of claim 1, wherein the pattern recognition comprises:
segmenting the enriched sensor data into time-windowed data blocks of a predefined duration;
computing, for each time-windowed data block, a feature vector that includes statistical measures of the sensor data and correlation measures between the sensor data and the medical data;
classifying each feature vector by applying a neural network classifier to produce classification data, wherein the classification data comprises a class label or probability scores corresponding to predefined categories related to the patient's health; and
generating activity pattern data that maps the classification data to the corresponding time-windowed data blocks, wherein the activity pattern data represents behaviors of the patient, including mobility, sleep patterns, and medication adherence, and serves as a baseline for detecting anomalies and predicting potential health risks.

3. The method of claim 2, wherein the neural network classifier is a deep learning model trained on historical sensor data and medical data to recognize patterns indicative of health conditions, and wherein the neural network classifier classifies produces classification data based on learned patterns of the patient's activity, enabling the detection of deviations from normal behavior.

4. The method of claim 1, wherein the room specific information provides insight into activity of the patient in different areas of a home.

5. The method of claim 1, further comprising creating a natural language summary by processing the pattern data, wherein the natural language summary is generated by a fine-tuned Large Language Model, the LLM model is fine-tuned based on historical sensor data and medical data to produce actionable insights related to patient care.

6. The method of claim 5, further comprising generating patient care instructions based on the natural language summary, the instructions specifying one or more actions to be performed to assist the patient.

7. The method of claim 6, wherein the patient care instructions are generated to specify a recommended course of action based on the natural language summary, the course of action being one of a situation advice to the patient, a modification of a treatment plan, a medication reminder, or an alert for medical assistance.

8. The method of claim 1, wherein the sensor data is received from the sensor network comprising motion sensors configured to detect patient movement, door sensors configured to record entry and exit times from different rooms, seating pressure sensors integrated into chairs configured to monitor sitting duration and frequency, and bed mat sensors configured to track sleep patterns, including sleep duration, restlessness and optionally heart rate data.

9. The method of claim 1, wherein the patient care instructions include recommendations based on historical health trends derived from medical data, the health trends including disease progression, symptom flare-ups, or treatment adherence.

10. The method of claim 1, wherein the delivery of the instructions is performed using at least one of a voice assistant and a mobile device.

11. The method of claim 1, further comprising storing the activity pattern data and the natural language summary in a database for future use and refinement of the prediction model.

12. The method of claim 1, wherein the instructions are delivered in real-time, the delivery being based on a dynamic analysis of the patient's current status as reflected in the most recent sensor and medical data.

13. The method of claim 1, further comprising authenticating a caregiver using electronic visit verification (EVV), wherein the EVV captures biometric authentication data, such as a fingerprint or facial recognition data, before delivery of the instruction data.

14. The method of claim 1, wherein the sensor data is received from a set of sensors in raw format, wherein each sensor provides its respective data, and wherein the sensor data from the set of sensors is mapped and synchronized into a standardized format, with each piece of standardized sensor data further including a time stamp associated with the corresponding sensor data.

15. The method of claim 1, wherein the medical data is prestored and the medical data is associated with the patient, the medical data comprising at least one of medical conditions, treatment history, medication data, and vital sign data.

16. A system comprising:
a sensor network configured to collect sensor data, in real time, comprising at least one of motion data, occupancy data, and environmental data, wherein each piece of sensor data is associated with a time stamp;
a memory configured to store medical data associated with a patient;
a processor operatively coupled to the sensor network and the memory, the processor configured to:
enrich the sensor data by incorporating room-specific information;
generate, in real time, activity pattern data by processing the enriched sensor data and the medical data using a pattern recognition model, wherein the activity pattern data comprises at least one of mobility, sleep patterns, and medication adherence, statistical measures of sensor data, temporal patterns, and correlations between sensor data and medical data;
detect an anomaly indicating a potential health risk by comparing the activity pattern data with baseline activity pattern data of the patient;
predict a health status of the patient based on the anomaly and the activity pattern data by utilizing a prediction model, wherein the prediction model is trained on data from other patients and medical data associated with the patient to recognize patterns correlated with health conditions;
generate patient care instructions based on the predicted health status of the patient; and
a secure communication network configured to deliver the patient care instructions to at least one of a patient interface device, a caregiver mobile application, or an automated medication dispensing system.

17. The system of claim 16, wherein the pattern recognition model:
segments the enriched sensor data into time-windowed data blocks of a predefined duration;
computes, for each time-windowed data block, a feature vector that includes statistical measures of the sensor data and correlation measures between the sensor data and the medical data;
classifies each feature vector by applying a neural network classifier to produce classification data, wherein the classification data comprises a class label or probability scores corresponding to predefined categories related to the patient's health; and
generates activity pattern data that maps the classification data to the corresponding time-windowed data blocks, wherein the activity pattern data represents behaviors of the patient, including mobility, sleep patterns, and medication adherence, and serves as a baseline for detecting anomalies and predicting potential health risks.

18. The system of claim 16, wherein the neural network classifier is a deep learning model trained on historical sensor data and medical data to recognize patterns indicative of health conditions, and wherein the neural network classifier classifier produces classification data based on learned patterns of the patient's activity, enabling the detection of deviations from normal behavior.

19. The system of claim 16, further comprising creating a natural language summary by processing the pattern data, wherein the natural language summary is generated by a fine-tuned Large Language Model, the LLM model is fine-tuned based on historical sensor data and medical data to produce actionable insights related to patient care.

20. The system of claim 16, wherein the sensor data is received from a set of sensors in raw format, wherein each sensor provides its respective data, and wherein the sensor data from the set of sensors is mapped and synchronized into a standardized format, with each piece of standardized sensor data further including a time stamp associated with the corresponding sensor data.
